(19) United States
(12) Patent Application Publication
(10) Pub. No.: US 2026/0000599 A1
(43) Pub. Date: Jan. 1, 2026

(54) PEEL-OFF LIP LINER STAIN

(71) Applicant: Sacheu Beauty Inc., Studio City, CA (US)

(72) Inventors: Quinn Roukema, Studio City, CA (US); Sarah Cheung, Los Angeles, CA (US)

(73) Assignee: Sacheu Beauty Inc., Studio City, CA (US)

(21) Appl. No.: 19/334,451

(22) Filed: Sep. 19, 2025

Related U.S. Application Data
(63) Continuation of application No. 18/675,021, filed on May 27, 2024.
(60) Provisional application No. 63/469,388, filed on May 27, 2023.

(51) Int. Cl. A61K 8/81; A61K 8/34; A61K 8/37; A61K 8/49; A61K 8/73; A61Q 1/04; A61Q 19/00

(52) U.S. Cl. CPC A61K 8/8129; A61K 8/345; A61K 8/375; A61K 8/498; A61K 8/735; A61K 8/8111; A61Q 1/04; A61Q 19/001

(57) ABSTRACT

A lip liner including a solvent configured to evaporate substantially independently of a user's skin, a film-forming agent dissolved in the solvent and configured to form a uniform film over the user's skin as the solvent evaporates, and one or more colorants dissolved in the solvent and configured to stain the user's skin.

### CROSS-REFERENCE TO RELATED APPLICATIONS

This application claims priority under 35 U.S.C. § 120 from U.S. patent application Ser. No. 18/675,021, filed May 27, 2024, which claims priority to U.S. Provisional application No. 63/469,388, filed on May 27, 2023 in the United States Patent and Trademark Office, the disclosures of which are incorporated herein in their entirety by reference.

### BACKGROUND

Lip liner is a cosmetic used to touch up the outer edges of a user's lips, often before applying lipstick or similar cosmetics, so that the user's lips will appear more even in shape, more defined, and also to help prevent smearing of lipstick beyond the borders of the user's lips.

Existing lip liners are made of waxes, oils, and colorants, and customarily are shaped as pencils in order to assist in drawing precise lines.

These existing products have many drawbacks. They wear off inconsistently over time, leading to the user's lips appearing splotchy. Furthermore these products can run or come off with use. This wear can also lead to inconsistent coloring on a user's lips, along with smearing, i.e., lip liner being smeared onto the user's face or fingers, as well as on other objects such as eating utensils or drinking glasses.

Many cosmetic products also use denatured alcohol, also called alcohol denat. as an antimicrobial and emulsifying agent for other ingredients. However, alcohol denat is not suitable for long-term use on skin. Alcohol denat may evaporate quickly, and as a result may draw out moisture from the user's skin, leaving the skin overly dry and causing irritation and damage to the skin's barrier. Furthermore, long-term use of products containing alcohol denat can dissolve the skin's surface sebum film, damaging the skin's protective barrier, reducing skin tolerance, and causing sensitivity and irritation. Alcohol denat is particularly undesirable for a user's lips, which have especially sensitive skin and so are more vulnerable to the damage alcohol denat may cause.

There is accordingly a need for a lip liner that addresses some or all of the above issues.

### SUMMARY OF THE INVENTION

Embodiments of the present general inventive concept provide a liquid lip liner configured to stain a user's lips a desired color, and a method of making the same.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a lip liner including a solvent configured to evaporate substantially independently of a user's skin, a film-forming agent dissolved in the solvent and configured to form a uniform film over the user's skin as the solvent evaporates, and one or more colorants dissolved in the solvent and configured to stain the user's skin a desired color.

In an embodiment the film-forming agent may be configured to form a plurality of filaments as the solvent evaporates, the plurality of filaments being configured to form the film over the user's skin. The film may be configured to be peeled off of the user's skin after the solvent has evaporated.

In an embodiment, the solvent may have a concentration by weight of about 40.4 percent.

In an embodiment, the film-forming agent may have a concentration by weight between about 5 percent and about 20 percent.

In an embodiment, the film-forming agent may include polyvinyl alcohol.

In an embodiment, the lip liner may further include a color fixing agent dissolved in the solvent and configured to enhance the dispersion and penetration of the one or mor colorants into the user's skin, the color fixing agent having a concentration by weight of about 15 percent.

In an embodiment, the color-fixing agent may include polyisobutene.

In an embodiment, the lip liner may further include a humectant dissolved in the solvent, the humectant being configured to absorb water from the environment, and a moisturizer dissolved in the solvent, the moisturizer being configured to form a moisture barrier over the user's skin to contain water.

In an embodiment, the solvent may be configured to evaporate from the user's skin between about 10 minutes and about 15 minutes after application thereto.

In an embodiment, the lip liner may further include a skin conditioning agent dissolved in the solvent.

In an embodiment, the skin conditioning agent may include diisostearyl malate dissolved in the solvent at a concentration of about 5.6 percent by weight.

In an embodiment, the lip liner may further include an antioxidant dissolved in the solvent.

In an embodiment, the antioxidant may include tocopherol dissolved in the solvent at a concentration of about 4.8 percent by weight.

In an embodiment, the lip liner may further include a preservative dissolved in the solvent at a concentration of about 0.1 percent by weight.

The foregoing and/or other features and utilities of the present general inventive concept may be achieved by providing a method of manufacturing a lip liner, the method including providing a solvent at a concentration of about 40.4 percent by weight, dissolving a film-forming agent in the solvent at a concentration between about 5 percent and about 20 percent by weight, and dissolving one or more colorants in the solvent at a concentration between about 0.2 percent and about 1.1 percent by weight.

In an embodiment, the method may further include comparing the combined solvent, film-forming agent, and one or more colorants with a color sample.

In an embodiment, the method may further include vacuum defoaming and filtering the combined film-forming agent, solvent, and one or more colorants.

In an embodiment, the method may further include dissolving a humectant into the solvent, the humectant being configured to absorb water from the environment, and dissolving a moisturizer into the solvent, the moisturizer being configured to form a moisture barrier over the user's skin to contain water.

### BRIEF DESCRIPTION OF THE DRAWINGS

These and/or other features and utilities of the present general inventive concept will become apparent and more readily appreciated from the following description of the embodiments, taken in conjunction with the accompanying drawings of which:

FIG. 1 illustrates a lip liner application according to an exemplary embodiment of the present general inventive concept;

FIG. 2 illustrates a process of manufacturing a lip liner according to an exemplary embodiment of the present general inventive concept; and

FIGS. 3A-3C illustrate a process of applying and subsequently peeling off a lip liner according to an exemplary embodiment of the present general inventive concept.

### DETAILED DESCRIPTION OF EMBODIMENTS OF THE INVENTIVE CONCEPT

Reference will now be made in detail to embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part “includes” or “comprises” an element, unless there is a particular description contrary thereto, the part can further include other elements, not excluding the other elements.

Hereinafter, one or more exemplary embodiments of the present general inventive concept will be described in detail with reference to accompanying drawings.

A lip liner 100 according to embodiments of the present general inventive concept may act as a stain, ingraining a desired color into a user's skin, for example the skin of the user's lips. The lip liner 100 may be applied to the user's skin as a liquid, and then be allowed to dry, in the process forming a film 110 on the user's skin. Once dry, the film 110 may be removed, leaving the user's skin stained the desired color. Because the stain is ingrained into the user's skin instead of being layered on over the skin, the color may last until it fades naturally, without smudging or coming off on other objects.

A lip liner 100 according to embodiments of the present general inventive concept may be specially formulated to be safe to use around the sensitive skin of the lip area. To this end, a lip liner 100 according to embodiments of the present general inventive concept may lack alcohol denat or any similar components. Embodiments may instead include components which avoid drying out or damaging the user's skin, and may instead nourish, moisturize, and heal the user's skin. For this reason, the lip liner 100 may be re-applied as desired to ensure the color on the user's skin remains consistent, without damaging or drying out the user's skin.

A lip liner 100 according to embodiments of the present general inventive concept may include a solvent such as water at a concentration of about 40.4% by weight. According to a preferred embodiment of the present general inventive concept, the solvent may be capable of evaporating in air at a standard air pressure of one atmosphere. The solvent may further be capable of evaporating substantially independently of the user's skin, meaning that as the solvent evaporates it does not damage the user's skin, and draws little to no moisture out of the skin. The solvent may assist in blending and dispersing other ingredients of the lip liner 100, serving as both a diluent and solvent within the lip liner 100.

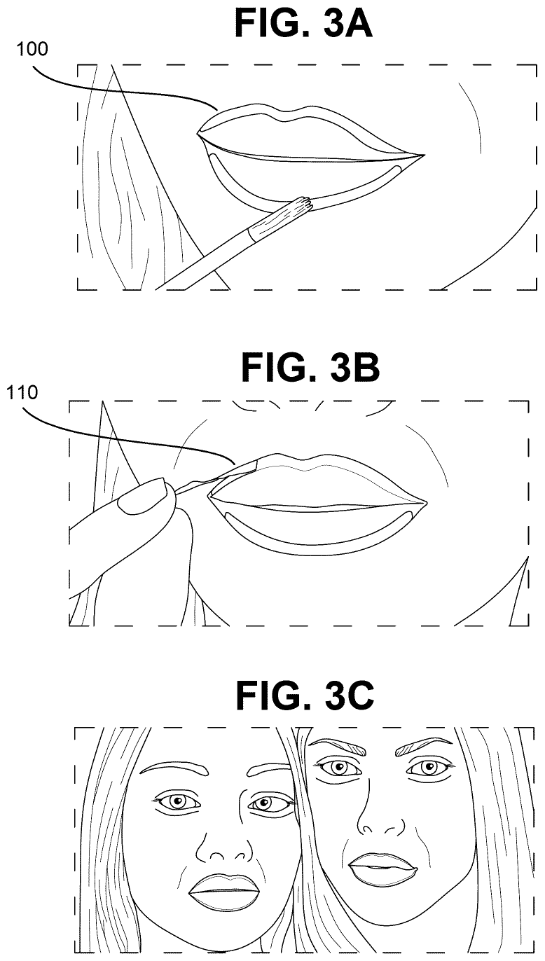

The lip liner 100 may further include a film-forming agent such as polyvinyl alcohol dissolved in the solvent at a concentration between 5% and 20% by weight. The film-forming agent may be selected from a group of materials capable of forming a physical film 110 over the surface of the lip liner 100 as the solvent evaporates in the air, as illustrated for example in FIG. 3B. The film-forming agent may form this film 110 without damaging or drawing moisture out of the user's skin. Once dried, the film 110 may be peeled away, according to a process described in greater detail below. Polyvinyl alcohol is a chemically synthesized polymer which may be used as the film-forming agent in a preferred embodiment of the present general inventive concept. After being applied to a user's skin, it may form filaments which come together to rapidly form a uniform, durable film 110 over the user's skin surface as the moisture in the lip liner 100, including moisture from the solvent, evaporates.

A preferred embodiment of the lip liner according to the present general inventive concept may further include one or more colorants, the colorant(s) being selected from a group of materials capable of penetrating the user's skin and staining it a desired color after the lip liner 100 is applied thereto, while the solvent evaporates and the film-forming agent forms the film 110. According to embodiments of the present general inventive concept, the colorant(s) may be water soluble, safe for human skin, non toxic, and non-bleeding, i.e., they have minimal “bleed” or flow beyond where they were applied to the user's skin. Concentration of the colorant(s) may vary depending on the desired color in a given embodiment of the present general inventive concept. According to embodiments of the present general inventive concept, the lip liner 100 may include one or more colorants, each at a concentration between about 0.2% by weight and 1.1% by weight.

A preferred embodiment of the lip liner 100 according to the present general inventive concept may further include a color-fixing agent such as polyisobutene at a concentration of about 15% by weight. For the purposes of this description, “color fixing agent” is a component which may enhance the dispersion and penetration of the colorant into the user's skin. Polyisobutene is a very stable, high-viscosity synthetic resin which may be used to provide adhesion. According to embodiments of the present general inventive concept, polyisobutene may be used to ensure that the lip liner 100 can evenly and completely cover the surface of the user's skin during application, avoiding uneven application or missed spots. Polyisobutene may therefore help to maintain the cohesiveness of the lip liner 100, and furthermore may ensure that the lip liner 100 does not shatter during application and drying. Furthermore, according to embodiments of the present general inventive concept, polyisobutene may be preferable to other color-fixing agents or adhesives, because polyisobutene may be extremely friendly to the user's skin, causing very low irritation. Still further, polyisobutene may not affect the performance or functions of other ingredients in the lip liner 100, making the entire formulation of the lip liner 100 more stable and effective.

A preferred embodiment of the lip liner 100 according to the present general inventive concept may further include additional components to provide color longevity, as well as moisturizers and emollients, which may soften or smooth a user's skin. Examples of these additional components include glycerin, hyaluronic acid, and diisostearyl malate. Glycerin may be included at a concentration between about 5% and about 10% by weight, and may serve as a moisturizer and also promote color longevity. Glycerin may function as a humectant, helping to attract and lock in moisture, thereby helping to maintain a moisture balance of the user's skin. The high-quality hydrating property of glycerin may help moisturize the user's skin, which may help the lip liner 100 apply more smoothly to the user's skin. Furthermore, glycerin may have hygroscopic properties, meaning it may absorb moisture from the air even in a dry environment. As such, glycerin may bring extra moisture to the user's skin, which may help to repair dry and cracked skin.

Hyaluronic acid may be a moisturizer included in the lip liner 100 at a concentration between about 0.1% and about 3% by weight. Hyaluronic acid is a natural polysaccharide present within the human body, and has exceptional moisture absorption capabilities, being able to absorb up to 1000 times its weight in water, and so may deeply hydrate and moisturize the user's skin, keeping the skin soft and eliminating dryness or cracking. The addition of hyaluronic acid may therefore enhance the lip liner 100's moisturizing performance, allowing a user's skin to get deep nourishment while obtaining color from the colorant in the lip liner 100.

According to embodiments of the present general inventive concept, an overall performance of the lip liner 100 may be adjusted according to the ratio of glycerin and hyaluronic acid, as shown in Example 3, below. Changing the concentration of an ingredient of the lip liner 100, such as hyaluronic acid for example, may change the effect and feel of the lip liner 100. At too low a concentration of hyaluronic acid, the lip liner 100 may not sufficiently moisturize the user's skin, potentially leading to dryness, discomfort, and ineffective product performance such as less lasting wear time or uneven color distribution. At a concentration of hyaluronic acid by weight of about 3%, the lip liner 100 may sufficiently moisturize the user's skin to avoid dryness or cracking, and may offer a desired consistency of the lip liner 100. Conversely at concentrations of hyaluronic acid over 3%, the lip liner 100 may cause adverse reactions such as bumpy texture on the user's skin, or can lead to product instability resulting in overflow, where the lip liner 100 does not adhere to the user's skin and instead seeps beyond the area of application.

Diisostearyl malate may be included in the lip liner 100 at a concentration of about 5.6% by weight. Diiosostearyl malate may be a skin conditioning agent and lubricant, and may moisturize and serve as an emollient, ensuring a smooth application and leaving a pleasant feel on the skin. Due to the emollient properties, diisostearyl malate may create a more stable and homogenous product by providing a better dispersion medium for the colorant(s). As a result, diisostearyl malate may promote color longevity, with color more evenly distributed on the user's skin.

A preferred embodiment of the lip liner 100 according to the present general inventive concept may also include tocopherol at a concentration of about 4.8% by weight. Tocopherol, which may also be known as “Vitamin E,” possesses antioxidant capabilities, and may protect the user's lips from the potential harm of pigments and environmental factors.

A preferred embodiment of the lip liner 100 according to the present general inventive concept may also include a preservative such as phenoxyethanol at a concentration of about 0.1% by weight. The preservative may ensure the lip liner 100 does not harbor bacteria or mold during its shelf life.

In a preferred embodiment of the present general inventive concept, the solvent may dissolve all the components of the lip liner 100 to form a homogenous mixture. In combination, the ingredients of the lip liner 100 may help the lip liner 100 provide an even protective layer on the user's skin, while also ensuring a natural coloration.

The combination of ingredients therefore may provide both a desirable makeup effect and nourishment to the user's skin.

According to exemplary embodiments of the present general inventive concept, the homogenous mixture of the lip liner 100 may have a liquid consistency. A user may apply this lip liner 100 to a desired portion of their skin with a precision applicator, e.g., a small brush, as illustrated for example in FIG. 3A.

Once exposed to air, the solvent in the lip liner 100 may begin to evaporate, causing the lip liner 100 to dry on the user's skin. During this drying time, colorants in the lip liner 100 may stain the user's skin, thereby imparting the desired color to the user's skin. Without alcohol denat, which encourages evaporation, a lip liner 100 according to embodiments of the present general inventive concept may take a relatively longer time to dry than a conventional lip liner 100, for example 10-15 minutes. This extended drying time may give relatively more time for the one or more colorants in the lip liner 100 to be imparted to the user's skin, resulting in a more consistent color, as well as a color that lasts longer by virtue of deeper penetration into the user's skin. While the lip liner 100 is drying, the film-forming agent may form a thin film 110 over the surface of the lip liner 100. This film 110 may adhere to the user's skin as the solvent evaporates fully, thereby ensuring that the lip liner 100 remains in place and only the skin the user originally applied the lip liner 100 to is stained. Once the lip liner 100 has been allowed to dry on the user's skin for a desired time to impart color, the film 110 may be peeled off of the user's skin, as illustrated for example in FIG. 3B. When the film 110 is peeled off, it exposes the user's skin, as well as the developed color on the skin, as illustrated for example in FIG. 3C. Removal of the film 110 may also remove any excess lip liner 100 which had not yet dried.

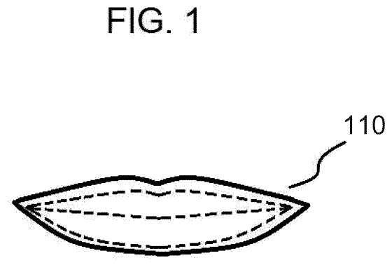

As illustrated in FIG. 1, the film 110 of lip liner 100 may help define a border around a desired area, for example a user's lips, which are illustrated in FIG. 1 as dashed lines. While the film 110 is in place, a user may apply cosmetic products, e.g., lipstick, to the desired area, and the film 110 may act as a border or barrier to prevent “bleed,” i.e., the cosmetic products extending beyond the area defined by the lip liner 100. According to an exemplary embodiment of the present general inventive concept, the film 110 formed by the lip liner 100 may catch any “bleed.” For example, lipstick extending past the user's lips may fall on the film 110 of the lip liner 100, and this bleed may be removed with the film 110. As such, a user may use the lip liner 100 to define an exact area where they want to apply cosmetic products, and may remove any bleed or errors in applying any such cosmetic products to the defined area.

It will be understood that the applications illustrated in FIG. 1 and FIGS. 3A-3C are only exemplary embodiments of the present general inventive concept. According to other exemplary embodiments, the lip liner 100 may be applied over a larger area, for example the entire surface of the user's lips, such that the lip liner 100 stains the user's skin the desired color. According to this exemplary embodiment, the lip liner 100 may be applied in lieu of other cosmetics, rather than in addition to them.

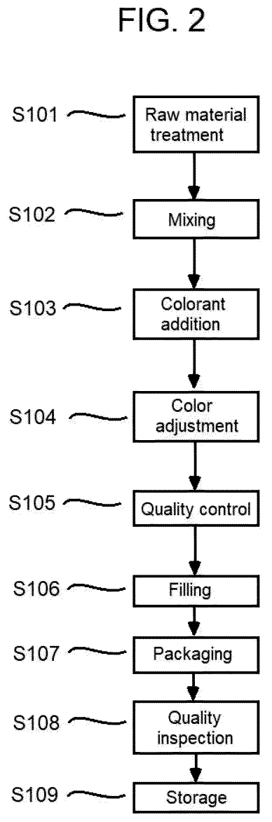

FIG. 2 illustrates a process of manufacturing a lip liner 100 according to an exemplary embodiment of the present general inventive concept. As illustrated therein, according to exemplary embodiments of the present general inventive concept, at step S101 of the process, raw materials, including the film-forming agent and solvent, may be treated with ultraviolet radiation to sterilize them and prepare them for processing.

At step S102 of the process, the treated raw materials may be added to a thermostatic stirring pot according to the formula, and may be stirred and melted uniformly at a constant temperature. According to exemplary embodiments of the present general inventive concept, the stirring pot may be made of stainless steel or other non-reactive material suitable for high temperatures.

Following step S102 of the process, the material may be substantially transparent and uniform, i.e., the various components may be thoroughly mixed together to be homogenous. Once the material has reached this state, in step S103 of the process one or more colorants may be weighed, melted, and added to the stirring pot according to formula requirements.

At step S104 of the process, the material with colorant added may be compared with an original sample to double-check the color. If a color difference is detected, additional colorants may be added to the material until the resulting material matches the original sample. At this point the colored material may be vacuum defoamed and filtered. This step may also be called “percolation.”

At step S105, the material may be inspected for quality, which may include inspecting the material for color, viscosity, total number of microorganisms, etc. After it is confirmed that the material meets preset quality requirements, the material may be stored and sterilized via ultraviolet radiation.

At step S106, the material may be filled and produced according to a desired order. According to an exemplary embodiment of the present general inventive concept, this filling and producing may comprise loading the processed material into one or more storage containers, for example a tube.

At step S107, the filled products may be packaged, i.e., the storage containers may be put into retail packaging for sale. At step S108 the packaged products may undergo a quality control inspection to detect any quality issues. Finally at step S109 the inspected packaged products may be put into storage, where they may await transport and delivery to a final consumer.

A lip liner according to embodiments of the present general inventive concept may allow users to impart a desired color to their skin without concerns of the color coming off or smearing with use, and also without risk of damaging or drying out their skin. Embodiments of the present general inventive concept may also allow a user to more precisely apply cosmetic products, avoiding bleed.

### Example 1

A lip liner was prepared with water as a solvent at a concentration of 40.4% by weight and polyvinyl alcohol as a film-forming agent at a concentration of 20% by weight. Polyisobutene as a color-fixing agent was added at a concentration of 15% by weight. Diisostearyl malate was added at a concentration of 5.6% by weight. Tocopherol was added at a concentration of 4.8% by weight. Phenoxyethanol was added at a concentration of 0.1% by weight. The colorants used were CI 16035 at a concentration of about 0.4% by weight, CI 45410 at a concentration of about 0.3% by weight, CI 19140 at a concentration of about 0.2% by weight, and CI 42090 at a concentration of about 0.2% by weight.

To prepare the lip liner, the ingredients were mixed in groups. The first ingredients added to the stirring pot were “Group A” ingredients, including the solvent and color-fixing agent, as well as diisostearyl malate and tocopherol. These Group A ingredients were added to the stirring pot, heated to a temperature of about 50 degrees Celsius, and stirred at a rate of about 100 rpm. The Group A ingredients were mixed under these conditions for about 20 minutes.

After the Group A ingredients were mixed, “Group B” ingredients were added to the stirring pot. “Group B” ingredients include the film-forming agent. Once the Group B ingredients were added, the stirring pot was heated to about 95 degrees Celsius and stirred at a rate of about 100 rpm. The combination of Group A and Group B was stirred under these conditions for about 30 minutes. The process of mixing and combining ingredient Groups A and B would take place during step S102, or “Mixing,” of the process illustrated in FIG. 2.

Once Group A and Group B ingredients were mixed together, they were mixed with “Group C ingredients,” which include the colorants and phenoxyethanol. The mixed Group A, B, and C ingredients were put into a sieving grinding machine and ground 1-3 times, depending on whether the colorants, which were powders, had been ground evenly. The process of adding Group C ingredients and grinding them would take place during step S103, or “Colorant Addition,” of the process illustrated in FIG. 2.

Following the grinding, the lip liner was completed according to the process illustrated in FIG. 2, after which it was ready for application. Once the lip liner was applied to the user's skin and allowed to dry, a process taking approximately 15 minutes, the film was peeled off of the user's skin. The film was solid enough to be removed as one piece. The lip liner successfully stained the user's skin the desired color.

### Example 2

A lip liner was prepared with identical formulation and process to Example 1, except it included polyvinyl alcohol at a concentration of 5% by weight. The lip liner was applied to the user's skin and allowed to dry. Drying time was similar to that of Example 1. The film was easier to remove from the user's skin than in Example 1, but the film was more fragile and did not come off smoothly in one piece. The lip liner still successfully stained the user's skin the desired color.

### Example 3

A lip liner was prepared with an identical formulation as Example 1, further including glycerin at a concentration of 10% by weight, and hyaluronic acid at a concentration of 3% by weight. The process for manufacture was identical to Example 1, except that hyaluronic acid was included in “Group A” ingredients, while glycerin was included in “Group B” ingredients.

It was found that the lip liner with this formulation provided both durable color and ample moisture retention, providing the user a comfortable feeling. There was minimal overflow or adverse reactions in the user's skin. Glycerin and hyaluronic acid were found to assist in each others' functions at these concentrations. Glycerin as a humectant pulled in water molecules from the environment, and hyaluronic acid acted as a “moisture barrier,” locking the water molecules in to prevent evaporation.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. A lip liner, comprising:
a solvent configured to evaporate substantially independently of a user's skin;
a film-forming agent dissolved in the solvent and configured to form a uniform film over the user's skin as the solvent evaporates;
one or more colorants dissolved in the solvent and configured to stain the user's skin a desired color.

2. The lip liner of claim 1, wherein the film-forming agent is configured to form a plurality of filaments as the solvent evaporates, the plurality of filaments being configured to form the film over the user's skin; and
the film is configured to be peeled off of the user's skin after the solvent has evaporated.

3. The lip liner of claim 1, wherein the solvent has a concentration by weight of about 40.4 percent.

4. The lip liner of claim 1, wherein the film-forming agent has a concentration by weight between about 5 percent and about 20 percent.

5. The lip liner of claim 1, wherein the film-forming agent comprises polyvinyl alcohol.

6. The lip liner of claim 1, further comprising a color fixing agent dissolved in the solvent and configured to enhance the dispersion and penetration of the one or more colorants into the user's skin, the color fixing agent having a concentration by weight of about 15 percent.

7. The lip liner of claim 6, wherein the color-fixing agent comprises polyisobutene.

8. The lip liner of claim 1, further comprising:
a humectant dissolved in the solvent, the humectant being configured to absorb water from the environment; and
a moisturizer dissolved in the solvent, the moisturizer being configured to form a moisture barrier over the user's skin to contain water.

9. The lip liner of claim 1, wherein the solvent is configured to evaporate from the user's skin between about 10 minutes and about 15 minutes after application thereto.

10. The lip liner of claim 1, further comprising a skin conditioning agent dissolved in the solvent.

11. The lip liner of claim 10, wherein the skin conditioning agent comprises diisostearyl malate dissolved in the solvent at a concentration of about 5.6 percent by weight.

12. The lip liner of claim 1, further comprising an antioxidant dissolved in the solvent.

13. The lip liner of claim 12, wherein the antioxidant comprises tocopherol dissolved in the solvent at a concentration of about 4.8 percent by weight.

14. The lip liner of claim 1, further comprising a preservative dissolved in the solvent at a concentration of about 0.1 percent by weight.

15. A method of manufacturing a lip liner, the method comprising:
providing a solvent at a concentration of about 40.4 percent by weight;
dissolving a film-forming agent in the solvent at a concentration between about 5 percent and about 20 percent by weight; and
dissolving one or more colorants in the solvent at a concentration between about 0.2 percent and about 1.1 percent by weight.

16. The method of claim 15, further comprising comparing the combined solvent, film-forming agent, and one or more colorants with a color sample.

17. The method of claim 15, further comprising vacuum defoaming and filtering the combined film-forming agent, solvent, and one or more colorants.

18. The method of claim 15, further comprising:
dissolving a humectant into the solvent, the humectant being configured to absorb water from the environment; and
dissolving a moisturizer into the solvent, the moisturizer being configured to form a moisture barrier over the user's skin to contain water.
